(19) United States
(12) Patent Application Publication
(10) Pub. No.: US 2023/0302021 A1
(43) Pub. Date: Sep. 28, 2023

(54) COMPOSITIONS AND METHODS FOR TREATING CASTRATION-RESISTANT PROSTATE CANCER

(71) Applicant: Purdue Research Foundation, West Lafayette, IN (US)

(72) Inventors: Kee-Hong KIM, West Lafayette, IN (US); Sora KIM, West Lafayette, IN (US); Shin KIM, New York, NY (US); Young KIM, New York, NY (US)

(21) Appl. No.: 18/187,866

(22) Filed: Mar. 22, 2023

Related U.S. Application Data
(60) Provisional application No. 63/322,698, filed on Mar. 23, 2022.

(51) Int. Cl. A61K 31/58; A61K 31/4166; A61K 31/18; A61P 35/00

(52) U.S. Cl. CPC A61K 31/58; A61K 31/4166; A61K 31/18; A61P 35/00

(57) ABSTRACT

This present disclosure teaches a method for treating or delaying castration-resistant prostate cancer (CRPC) comprising co-administering to a subject in need of relief from said cancer a therapeutically effective amount of a sterol-O-acyltransferase 1 (SOAT1) inhibitor together with a therapeutically effective amount of one or more androgen receptor inhibitors/antagonists. Examples of inhibitor/antagonist of androgen receptor and androgen biosynthesis include enzalutamide and abiraterone; and examples of SOAT1 inhibitors include avasimibe. Pharmaceutical compositions and methods of uses for the treatment of CRPCs are within the scope of this disclosure.

### CROSS-REFERENCE TO RELATED APPLICATION

The present application claims priority from U.S. Provisional Application No. 63/322,698, filed on Mar. 23, 2022, which is incorporated herein by reference in its entirety.

### FIELD OF THE INVENTION

The present invention relates to a novel strategy for treating castration-resistant prostate cancer by using a sterol O-acyltransferase (SOAT) inhibitor [or acyl-CoA:cholesterol acyltransferase (ACAT) inhibitor] in combination with the therapeutic agent.

### BACKGROUND

This section introduces aspects that may help facilitate a better understanding of the disclosure. Accordingly, these statements are to be read in this light and are not to be understood as admissions about what is or is not prior art.

Current prostate cancer therapies include targeting androgen signaling and also include androgen deprivation therapy by administration of anti-androgen drugs and androgen antagonists to patients with prostate cancer. These methods may be effective at first, but often become ineffective if the patient develops castration-resistant prostate cancer (CRPC). To date, CRPC is not curable. CRPC patients and patients whose docetaxel treatment does not improve health are often treated with anti-androgen drugs such as enzalutamide (or a salt thereof) and abiraterone (or a salt thereof). However, drug resistance to such anti-androgen drugs often develops in prostate cancer patients. There is a growing need for new compositions and methods for treating or delaying anti-androgen drug-resistant CRPC. Herein, the present invention provides, in part, new compositions and methods for treating these CRPC patients.

Cholesteryl esters (CEs) are the storage form of free cholesterol. SOAT1 and SOAT2 catalyze the esterification of free cholesterol to CEs. SOAT1 is expressed in various tissues, while SOAT2 is mainly expressed in the liver and intestine. Both enzymes utilize long-chain fatty acyl-CoAs and sterols, such as cholesterol and various oxysterols, as their substrates. Accumulation of CEs has been shown to be a hallmark of prostate cancer progression. High SOAT1 protein expression has been demonstrated to correlate with a worse prognosis in prostate cancer. The methods of the invention include administration of SOAT inhibitor compounds for treating prostate cancer, e.g., drug-resistant prostate cancer, such as anti-androgen drug (e.g., enzalutamide and abiraterone) resistance and/or CRPC that is related to an accumulation of CEs. Herein, a “SOAT inhibitor” is an agent that inhibits SOAT1 and/or SOAT2 enzyme activities. Reported SOAT inhibitors include avasimibe (CI-1011), CI-976, CP113,818, pactimibe, NTE-122, F-1394, PD140296, PD128042, PD132301-2, octimibate, DuP128, 58-035, HL-004, SMP-500, CL-277,082, SKF-99085, CS-505, eflucimibe (F12511), E5324, FR145237, CL277,082, YM-17E, and FR129169. SOAT1 inhibitors that exhibit an IC₅₀ value for SOAT1 that is at least twice or higher than the corresponding IC₅₀ value for SOAT2, include K-604, pyrocarbonate, beauveriolides I, and methanol extracts of Saururus chinensis root containing saucerneol B and manassantin B. Inhibitors that inhibit both SOAT1 and SOAT2 include, for example, avasimibe (CI-1011), CI-976 and pactimibe. Avasimibe is an oral SOAT non-selective inhibitor with approximately similar potency (IC₅₀ of 24 μM for SOAT1 and IC₅₀ of 9.2 μM for SOAT2). Avasimibe is considered safe when administered to rats, dogs, and humans.

### SUMMARY OF THE INVENTION

An aspect of the present invention provides a method for treating or delaying castration-resistant prostate cancer (CRPC) comprising co-administering to a subject in need of relief from said cancer a therapeutically effective amount of a sterol-O-acyltransferase 1 (SOAT1) inhibitor together with a therapeutically effective amount of one or more inhibitors/antagonists of androgen receptor or androgen biosynthesis.

In some embodiments, the androgen receptor inhibitor/antagonist may comprise enzalutamide, bicalutamide, apalutamide, flutamide, nilutamide, darolutamide, and clascoterone, or a pharmaceutically acceptable salt thereof.

In some embodiments, the androgen receptor inhibitor/antagonist may be enzalutamide, or a pharmaceutically acceptable salt thereof.

In some embodiments, the androgen biosynthesis inhibitor/antagonist may be abiraterone, or a pharmaceutically acceptable salt thereof.

In some embodiments, the CRPC may be an androgen receptor inhibitor-resistant CRPC. For example, the CPRC may be an enzalutamide-resistant CRPC.

In some embodiments, the CRPC may be an androgen biosynthesis antagonist-resistant CRPC. For example, the CRPC may be an abiraterone-resistant CRPC.

In some embodiments, the SOAT1 inhibitor may comprise avasimibe (CI-1011), CI-976, CP113,818, pactimibe, NTE-122, F-1394, PD140296, PD128042, PD132301-2, octimibate, DuP128, 58-035, HL-004, SMP-500, CL-277,082, SKF-99085, CS-505, eflucimibe (F12511), E5324, FR145237, CL277,082, YM-17E, FR129169, K-604, pyrocarbonate, beauveriolides I, and methanol extracts of Saururus chinensis root containing saucerneol B and manassantin B, or a pharmaceutically acceptable salt thereof.

In some embodiments, the SOAT1 inhibitor may be avasimibe, or a pharmaceutically acceptable salt thereof.

In some embodiments, the inhibitor/antagonist of androgen receptor or androgen biosynthesis and the SOAT1 inhibitor may be combined first and administered consequently.

In some embodiments, the inhibitor/antagonist of androgen receptor or androgen biosynthesis and the SOAT1 inhibitor may be formulated separately and administered consequently.

Another aspect of the present invention provides a pharmaceutical composition comprising a SOAT1 inhibitor and an inhibitor/antagonist of androgen receptor or androgen biosynthesis, together with one or more of pharmaceutically acceptable diluents, excipients, or carriers.

In some embodiments, the androgen receptor inhibitor/antagonist may comprise apalutamide, flutamide, nilutamide, darolutamide, and clascoterone, or a pharmaceutically acceptable salt thereof.

In some embodiments, the androgen receptor inhibitor/antagonist may be enzalutamide, or a pharmaceutically acceptable salt thereof.

In some embodiments, the androgen biosynthesis inhibitor/antagonist may be abiraterone, or a pharmaceutically acceptable salt thereof.

In some embodiments, the CRPC may be an androgen receptor inhibitor-resistant CRPC. For example, the CPRC may be an enzalutamide-resistant CRPC.

In some embodiments, the CRPC may be an androgen biosynthesis antagonist-resistant CRPC. For example, the CRPC may be an abiraterone-resistant CRPC. I

In some embodiments, the SOAT1 inhibitor may comprise avasimibe (CI-1011), CI-976, CP113,818, pactimibe, NTE-122, F-1394, PD140296, PD128042, PD132301-2, octimibate, DuP128, 58-035, HL-004, SMP-500, CL-277,082, SKF-99085, CS-505, eflucimibe (F12511), E5324, FR145237, CL277,082, YM-17E, FR129169, K-604, pyrocarbonate, beauveriolides I, and methanol extracts of Saururus chinensis root containing saucerneol B and manassantin B, or a pharmaceutically acceptable salt thereof.

In some embodiments, the SOAT1 inhibitor may be avasimibe, or a pharmaceutically acceptable salt thereof.

Each embodiment disclosed herein is contemplated as being applicable to each of the other disclosed embodiments. Thus, all combinations of the various elements described herein are within the scope of the invention.

### BRIEF DESCRIPTION OF THE DRAWINGS

FIG. 1 is a line graph showing the effect of control, enzalutamide (ENZ), avasimibe (AVA), and combination of ENZ and AVA on the proliferation of 22RV1 cells.

FIG. 2A is an image showing the effect of ENZ, AVA, and a combination of ENZ and AVA on the colony formation of 22RV1 cells.

FIG. 2B is a bar graph showing the quantified colony formation shown in FIG. 2A.

FIG. 3A is an image showing the effect of ENZ, AVA, and a combination of ENZ and AVA on the colony formation of ENZ naïve C4-2B cells.

FIG. 3B is an image showing the effect of ENZ, AVA and a combination of ENZ and AVA on the colony formation of ENZ-resistant C4-2B cells.

FIG. 4 is a table showing the combination index values of ENZ in combination with AVA in 22RV1 cells.

FIG. 5A is a bar graph showing the efficacy of SOAT1 shRNA to knockdown the SOAT1 expression in 22RV1 cells.

FIG. 5B is a line graph showing the effect of control shRNA (shCTRL)+DMSO, shCTRL+ENZ, shSOAT1+DMSO and shSOAT1+ENZ on the proliferation of 22RV1 cells.

FIG. 6A is an image showing the effect of ENZ on the colony formation of shCTRL or shSOAT1 transfected 22RV1 cells.

FIG. 6B is a bar graph showing the quantified colony formation shown in FIG. 6A.

FIG. 7A is an image showing 22RV1-derived xenograft mice treated with ENZ, AVA, and a combination of ENZ and AVA.

FIG. 7B is a line graph showing the quantified tumor size shown in FIG. 7A.

FIG. 8 is a bar graph showing the tumor weight of 22RV1-derived xenograft mice treated with ENZ, AVA, and a combination of ENZ and AVA.

FIG. 9 is a bar graph showing the bodyweight of 22RV1-derived xenograft mice treated with ENZ, AVA, and a combination of ENZ and AVA.

FIGS. 10A-10E. Gene expression of AR signaling pathway is positively correlated with SOAT1 expression. (FIG. 10A) RNA-seq data of patients who had undergone hormone therapy were merged and divided into two groups based on SOAT1 gene expression for further analyses (FIG. 10B) Clusterprofiler analysis of differentially expressed genes show top 10 KEGG gene set enrichments. (FIG. 10C) GSEA confirms that AR signaling pathway gene set is enriched in high SOAT1 expressing group. (FIG. 10D) GSEA of Hallmark gene sets show potential pathways associated with SOAT1 expression (FIG. 10E) qPCR results of genes related to AR signaling

### DETAILED DESCRIPTION OF THE INVENTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure the term “about” can allow for a degree of variability in a value or range, for example, within 10%, within 5%, or within 1% of a stated value or of a stated limit of a range.

In the present disclosure the term “substantially” can allow for a degree of variability in a value or range, for example, within 90%, within 95%, or within 99% of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

An aspect of the present invention provides a method for treating or delaying enzalutamide-resistant CRPC. The method comprises co-administering to a subject in need a therapeutically effective amount of a SOAT inhibitor (or a pharmaceutically acceptable salt thereof) and a therapeutically effective amount of an anti-cancer therapeutic agent. In some embodiments, the method comprises co-administering to a subject in need a composition containing a therapeutically effective amount of a SOAT inhibitor (or a pharmaceutically acceptable salt thereof) and a composition containing a therapeutically effective amount of an anti-cancer therapeutic agent.

As used herein, the term “SOAT inhibitor” or “ACAT inhibitor” means avasimibe (CI-1011), K-604, CI-976, CP113,818, pactimibe, NTE-122, F-1394, PD140296, PD128042, PD132301-2, octimibate, DuP128, 58-035, HL-004, SMP-500, CL-277,082, SKF-99085, CS-505, eflucimibe (F12511) and F12511 analogs (analogs 1, 2, 2c and 3 or F26) (US2006/0135785), E5324, FR145237, CL277,082, YM-17E, FR129169, diethyl pyrocarbonate (Cho, et al. 2003, Biochem. Biophys. Res. Comm. 309:864-872), beauveriolides I and beauveriolides III (Oshiro, et al. 2007, J. Antibiotics 60:43-51), beauveriolide analogs (258, 274, 280, 285 and 301) (Tomoda & Doi, 2008, Accounts Chem. Res. 41:32-39), Compound 1A and its derivatives (1B, 1C and 1D) (Lada, et al. 2004, J. Lipid Res. 45:378-386), methanol extracts of Saururus chinensis root containing saucerneol B and manassantin B (Lee, et al. 2004, Bioorg. Med. Chem. Lett. 14:3109-3112), and derivatives of anilidic, ureidic or diphenyl imidazole compounds (PCT/US2014/054917).

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “subject” or “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fishes and the like.

As used herein, the term “administration” or “administering” of the subject compound refers to providing a compound of the invention and/or a prodrug thereof to a subject in need of treatment.

As used herein, the term “effective amount” or “therapeutically effective amount” refer to a sufficient amount of an active ingredient(s) described herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose-escalation study. By way of example only, a therapeutically effective amount of a compound of the invention may be in the range of e.g., about 0.01 mg/kg/day to about 1000 mg/kg/day, from about 0.1 mg/kg/day to about 500 mg/kg/day, from about 0.1 mg (×2)/kg/day to about 500 mg (×2)/kg/day.

In addition, such compounds and compositions may be administered singly or in combination with one or more additional therapeutic agents. The methods of administration of such compounds and compositions may include, but are not limited to, intravenous administration, inhalation, oral administration, rectal administration, parenteral, intravitreal administration, subcutaneous administration, intramuscular administration, intranasal administration, dermal administration, topical administration, ophthalmic administration, buccal administration, tracheal administration, bronchial administration, sublingual administration or optic administration. Compounds provided herein may be administered by way of known pharmaceutical formulations, including tablets, capsules or elixirs for oral administration, suppositories for rectal administration, sterile solutions or suspensions for parenteral or intramuscular administration, lotions, gels, ointments or creams for topical administration, and the like. In some embodiments, such pharmaceutical compositions are formulated as tablets, pills, capsules, a liquid, an inhalant, a nasal spray solution, a suppository, a solution, a gel, an emulsion, an ointment, eye drops, or ear drops.

The therapeutically effective amount may vary depending on, among others, the disease indicated, the severity of the disease, the age and relative health of the subject, the potency of the compound administered, the mode of administration and the treatment desired. The required dosage will also vary depending on the mode of administration, the particular condition to be treated and the effect desired.

The compounds described herein include all stereoisomers, geometric isomers, tautomers, isotopes, and prodrug of the structures depicted. The compounds described herein can be present in various forms including crystalline, powder and amorphous forms of those compounds, pharmaceutically acceptable salts, including, for example, polymorphs, pseudopolymorphs, solvates, hydrates, unsolvated polymorphs (including anhydrates), conformational polymorphs, and amorphous forms of the compounds, as well as mixtures thereof.

As used herein, the term “pharmaceutically acceptable” refers a material, such as a carrier or diluent, which does not abrogate the biological activity or properties of the compounds described herein. Such materials are administered to an individual without causing undesirable biological effects or interacting in a deleterious manner with any of the components of the composition in which it is contained.

As used herein, the term “pharmaceutically acceptable salt” refers to a formulation of a compound that does not cause significant irritation to an organism to which it is administered and does not abrogate the biological activity and properties of the compounds described herein.

Pharmaceutically acceptable salt forms may include pharmaceutically acceptable acidic/anionic or basic/cationic salts (UK Journal of Pharmaceutical and Biosciences Vol. 2(4), 01-04, 2014, which is incorporated herein by reference). Pharmaceutically acceptable acidic/anionic salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphosphate, polygalacturonate, salicylate, stearate, subacetate, succinate, sulfate, hydrogensulfate, tannate, tartrate, teoclate, tosylate, and triethiodide salts. Pharmaceutically acceptable basic/cationic salts include, the sodium, potassium, calcium, magnesium, diethanolamine, N-methyl-D-glucamine, L-lysine, L-arginine, ammonium, ethanolamine, piperazine, and triethanolamine salts.

A pharmaceutically acceptable acid addition salt of a compound of the invention may be prepared by methods known in the art and may be formed by reaction of the free base form of the compound with a suitable inorganic or organic acid including, but not limited to, hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, and hexanoic acid. A pharmaceutically acceptable acid addition salt can comprise or be, for example, a hydrobromide, hydrochloride, sulfate, bisulfate, nitrate, phosphate, succinate, maleate, formarate, acetate, propionate, fumarate, citrate, tartrate, lactate, benzoate, carbonate, benzathine, chloroprocaine, choline, histidine, meglumine, meglumine, procaine, triethylamine, besylate, decanoate, ethylenediamine, salicylate, glutamate, aspartate, p-toluenesulfonate, benzenesulfonate, methanesulfonate, ethanesulfonate, naphthalenesulfonate (e.g., 2-naphthalenesulfonate), and hexanoate salt.

A pharmaceutically acceptable base addition salt of a compound of the invention may also be prepared by methods known in the art and may be formed by the reaction of the free base form of the compound with a suitable inorganic or organic base including, but not limited to, hydroxide or other salt of sodium, potassium, lithium, ammonium, calcium, magnesium, iron, zinc, copper, manganese, aluminum, tromethamine, glycolate, hydrabamine, methylbromide, methylnitrate, octanoate, oleate, and the like.

A free acid or free base form of a compound of the invention may be prepared by methods known in the art (e.g., for further details see L. D. Bigley, S. M. Berg, D. C. Monkhouse, in “Encyclopedia of Pharmaceutical Technology”. Eds, J. Swarbrick and J. C. Boylam, Vol 13, Marcel Dekker, Inc., 1995, pp. 453-499, which is incorporated herein by reference). For example, a compound of the invention in an acid addition salt form may be converted to the corresponding free base form by treating with a suitable base (e.g., ammonium hydroxide solution, sodium hydroxide, and the like). A compound of the invention in a base addition salt form may be converted to the corresponding free acid by treating with a suitable acid (e.g., hydrochloric acid, etc.).

Aspects of this disclosure include prodrug forms of any of the compounds described herein. Any convenient prodrug forms of the subject compounds can be prepared, for example, according to the strategies and methods described by Rautio et al. (“Prodrugs: design and clinical applications”, Nature Reviews Drug Discovery 7, 255-270 (February 2008)).

Prodrug derivatives of the compounds of the invention may be prepared by methods known to those of ordinary skill in the art (e.g., for further details see Saulnier et al., Bioorg. Med. Chem. Letters, 1994, 4, 1985, which is incorporated herein by reference). Protected derivatives of the compounds of the invention may be prepared by means known to those of ordinary skill in the art. A detailed description of techniques applicable to the creation of protecting groups and their removal can be found in T. W. Greene, “Protecting Groups in Organic Chemistry,” 3rd edition, John Wiley and Sons, Inc., 1999 and “Design of Prodrugs”, ed. 11. Bundgaard, Elsevier, 1985, which are incorporated herein by reference.

The compounds of the present disclosure may be prepared as stereoisomers. Where the compounds have at least one chiral center, they may exist as enantiomers. Where the compounds possess two or more chiral centers, they may exist as diastereomers. The compounds of the invention may be prepared as racemic mixtures. Alternatively, the compounds of the invention may be prepared as their individual enantiomers or diastereomers by reaction of a racemic mixture of the compound with an optically active resolving agent to form a pair of diastereo-isomeric compounds, separating the diastereomers, and recovering the optically pure enantiomers. Resolution of enantiomers may be carried out using covalent diastereomeric derivatives of the compounds of the invention, or by using dissociable complexes (e.g., crystalline diastereomeric salts). Diastereomers have distinct physical properties (e.g., melting points, boiling points, solubility, reactivity, etc.) and may be readily separated by taking advantage of these dissimilarities. The diastereomers may be separated by chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet and Samuel H. Wilen, “Enantiomers, Racemates and Resolutions” John Wiley And Sons, Inc., 1981, which is incorporated herein by reference.

The compounds of the invention may be prepared as solvates (e.g., hydrates). The term “solvate” refers to a complex of variable stoichiometry formed by a solute (for example, a compound of the invention or a pharmaceutically acceptable salt thereof) and a solvent. Such solvents for the purpose of the invention may not interfere with the biological activity of the solute. Non-limiting examples of suitable solvents include water, acetone, methanol, ethanol and acetic acid. Preferably the solvent used is a pharmaceutically acceptable solvent.

Furthermore, the compounds of the invention may be prepared as crystalline forms. The crystalline forms may exist as polymorphs.

It should be noted that in view of the close relationship between the compound of the invention and their other forms, whenever a compound is referred to in this context herein, a corresponding salt, diastereomer, enantiomer, racemate, crystalline, polymorph, prodrug, hydrate, or solvate is also intended, if it is possible or appropriate under certain circumstances.

Another aspect of the present invention provides a composition for treating or delaying enzalutamide-resistant CRPC. The composition comprises a therapeutically effective amount of an SOAT inhibitor (or a pharmaceutically acceptable salt thereof) and a therapeutically effective amount of an anti-cancer therapeutic agent.

As used herein, the term “composition” is intended to encompass a product comprising the claimed compound, salt, diastereomer, enantiomer, racemate, hydrate, solvate, or a pharmaceutical combination thereof in the therapeutically effective amount, as well as any other product which results, directly or indirectly, from claimed compound, salt, diastereomer, enantiomer, racemate, hydrate, solvate, or a pharmaceutical combination thereof.

As used herein, the term “pharmaceutical composition” refers to a mixture of a therapeutically active component (ingredient) with one or more other components, which may be chemically or biologically active or inactive. Such components may include, but not limited to, carriers, stabilizers, diluents, dispersing agents, suspending agents, thickening agents, excipients, and adjuvants.

As used herein, the term “pharmaceutical combination” means a product that results from the mixing or combining of more than one therapeutically active ingredient.

As used herein, the term “acceptable” with respect to a formulation, composition or ingredient, as used herein, means having no persistent detrimental effect on the general health of the subject being treated.

As used herein, the term “carrier” refers to chemical or biological material that can facilitate the incorporation of a therapeutically active ingredient(s) into cells or tissues.

Suitable excipients may include, for example, water, pharmaceutically acceptable organic solvents such as paraffins (e.g., petroleum fractions), vegetable oils (e.g. groundnut or sesame oil), mono- or polyfunctional alcohols (e.g., ethanol or glycerol), carriers such as natural mineral powders (e.g., kaoline, clays, talc, chalk), synthetic mineral powders (e.g., highly dispersed silicic acid and silicates), sugars (e.g., cane sugar, lactose and glucose), emulsifiers (e.g., lignin, spent sulphite liquors, methylcellulose, starch and polyvinylpyrrolidone), and lubricants (e.g., magnesium stearate, talc, stearic acid and sodium lauryl sulphate).

Any suitable pharmaceutically acceptable carriers, stabilizers, diluents, dispersing agents, suspending agents, thickening agents, excipients, and adjuvants known to those of ordinary skill in the art for use in pharmaceutical compositions may be selected and employed in the compositions described herein. The compositions described herein may be in the form of a solid, liquid, or gas (aerosol). For example, they may be in the form of tablets (coated tablets) made of, for example, collidone or shellac, gum Arabic, talc, titanium dioxide or sugar, capsules (gelatin), solutions (aqueous or aqueous-ethanolic solution), syrups containing the active substances, emulsions or inhalable powders (of various saccharides such as lactose or glucose, salts and mixture of these excipients with one another), and aerosols (propellant-containing or -free inhale solutions). Also, the compositions described herein may be formulated for sustained or slow release.

Other embodiments and uses will be apparent to one skilled in the art in light of the present disclosures. The following examples are provided merely as illustrative of various embodiments and shall not be construed to limit the invention in any way.

### EXAMPLES

The invention is described in greater detail by the following non-limiting examples.

### Example 1: Material and Methods

Cell culture and drugs: C4-2B cells were maintained in RPMI 1640 supplemented with 10% fetal bovine serum (FBS), 100 units/ml penicillin and 100 units/ml streptomycin. 22Rv1 cells were maintained in RPMI 1640 supplemented with 10% fetal bovine serum (FBS), 100 units/ml penicillin and 0.1 mg/ml streptomycin supplemented with 1 mM sodium pyruvate and 10 mM HEPES buffer. ENZ-resistant C4-2B MDVR cells were maintained in 20 μM ENZ-containing medium. Cells were maintained at 37° C. in a humidified incubator with 5% carbon dioxide.

Lentiviral shRNA-mediated knockdown of SOAT1: Plasmids encoding shRNA for human SOAT1 (Sigma) and control shRNA were extracted with GeneJET Plasmid Midiprep Kit (Thermo Scientific) and lentivirus particles were produced. Briefly, 293T cells were transfected with 4.6 μg of the pLKO.1-target (pLKO.1-ACAT1, or pLKO.1-CTRL), 5.6 μg of pMLDg/pRRE, 2.6 μg of pRSV-rev, and 4.1 μg of pVSV-g using FuGENE (Promega). After 24 h, fresh 10% FBS-DMEM was applied to the transfected cells and the virus medium was then collected 24 h and 28 h later. Virus particles were harvested by filtration (0.45 μm pore size). The harvested viruses were employed to infect 22RV1 in the presence of 8 μg/ml Polybrene to express SOAT1 shRNA and silence their target gene expression via RNA interference. Finally, successfully transfected cells were selected by 3 μg/ml puromycin and maintained in 0.1 μg/ml for various analyses. Gene knockdown efficiency was determined by a real-time PCR assay.

Cell viability assay: An MTT assay was performed to evaluate the effect of drugs on cell viability. Ten thousand cells per well were plated in a 96-well plate and treated with drugs the following day. The MTT solution (0.5 mg/ml) was then applied to the cells for 1 h at 37° C. with 5% CO2. Precipitated formazan was dissolved in DMSO and quantified at 570 nm using a microplate reader (Beckman-Coulter).

Clonogenic assay: Cells were seeded with equal density in a 6-well plate and treated with DMSO or different drugs with designated concentrations for 14 days. The medium was changed every 4 days. After the colonies were fixed by 4% formaldehyde and stained with 5% crystal violet, colony numbers were determined by ImageJ.

Combination index: The data obtained from the MTT assay were used to calculate cell viability and also used to calculate combination index (CI). The Chou-Talalay method was applied to determine the CI using CompuSyn software (Biosoft). The interaction of the two drugs was primarily identified by the CI values, where CI<1 demonstrates synergism, CI=1 indicates an additive effect, and CI>1 indicates antagonism.

Real-time quantitative PCR: Total RNA was extracted by TriZol reagent. cDNAs were synthesized using M-MuLV Reverse Transcriptase (New England Biolabs). Real-time PCR with SYBR Green qPCR Master Mix (MedChemExpress) was used to quantify gene expression by StepOne real-time PCR system (Applied Biosystems). Data were normalized and analyzed using the ΔΔCt method.

22RV1-derived mouse xenograft model: All procedures were approved by the Purdue Animal Care and Use Committee. Five-week-old male nude mice (Jackson Laboratory) were purchased for the experiment. For xenograft, 22RV1 cells (2×10⁶ cells/mouse) were suspended in 100 μl of RPMI1640 media with 50% Matrigel matrix (BD Biosciences) and injected subcutaneously in the right dorsal flank of the nude mice 10 days after surgical castration. ENZ stock prepared in DMSO was diluted in corn oil for oral gavage and AVA solution was prepared. Once the tumors reached approximately 50-100 mm³, the mice were randomly divided into four groups for daily drug administration for 3 weeks: (i) vehicle oral gavage (corn oil)+vehicle intraperitoneal injection (2% tween-80 (v/v) and 3.2 mg/mL 2-hydroxypropyl-β-cyclodextrin in PBS); (ii) ENZ alone (25 mg/kg body weight/day) by oral gavage; (iii) AVA alone (20 mg/kg body weight/day) by intraperitoneal injection; (iv) ENZ (25 mg/kg/day)+AVA (20 mg/kg/day). Tumors were monitored every two days with a caliper and tumor volumes were calculated with the formula V=0.5×L×W² (where V is volume, L is length, W is the width). At the end of the study, mice were euthanized and tumor tissues were excised and weighted.

It was reported that ENZ-resistant cells, including 22RV1 cells are cross-resistant to abiraterone (Liu et al. 2016, Oncotarget, 7:32210-32220; Simon et al., Cancers, 2021 Mar. 23; 13(6):1483). Both ENZ and abiraterone target androgen pathway, in that ENZ acts as an androgen receptor inhibitor (Tran et al. 2009, Science, 324:787-790), and abiraterone is a potent and irreversible cytochrome inhibitor that inhibits androgen synthesis and acts as an androgen receptor antagonist (Richards et al. 2012, Cancer Res. 72:2176-2182). This indicates that AVA can sensitize not only ENZ-resistant but also abiraterone-resistant PCRC cells.

Statistical analysis: All data are presented as mean±SEM. Statistical analyses were performed with GraphPad Prism 5 program (GraphPad Software, Inc.). Two-tailed Student t-tests or ANOVA followed by Tuckey post hoc test were performed to analyze the statistical significance of the results. P<0.05 was considered statistically significant.

### Example 2: Pharmacological Inhibition of SOAT1 by AVA Sensitized Prostate Cancer Cells to ENZ

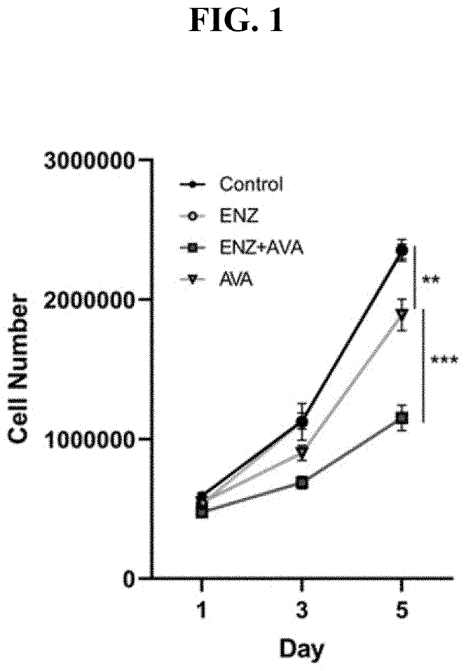

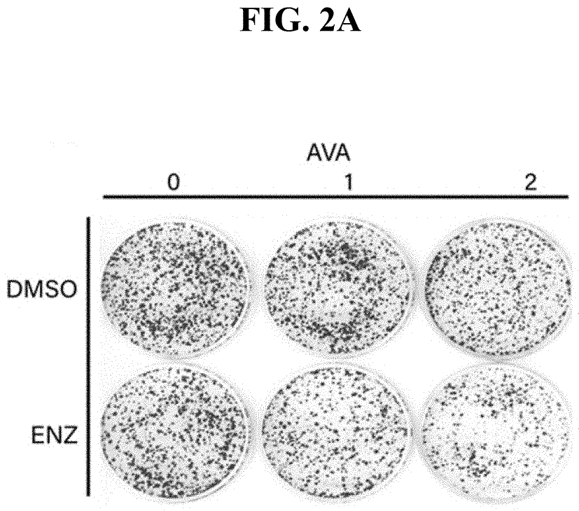

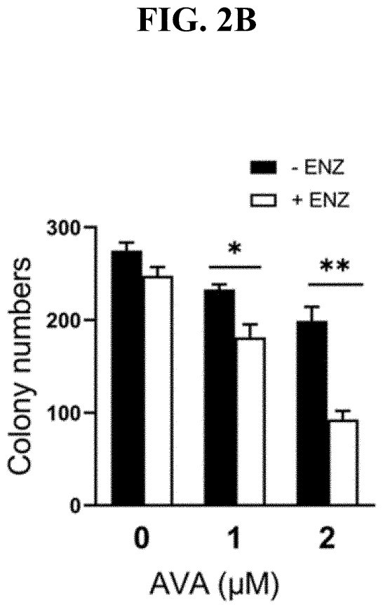

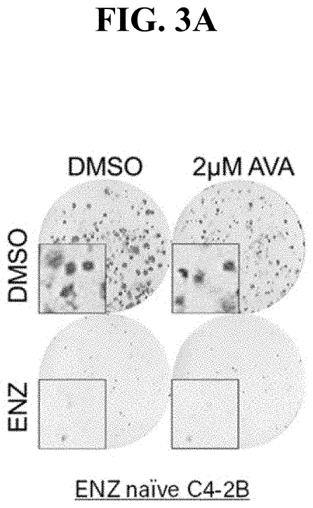

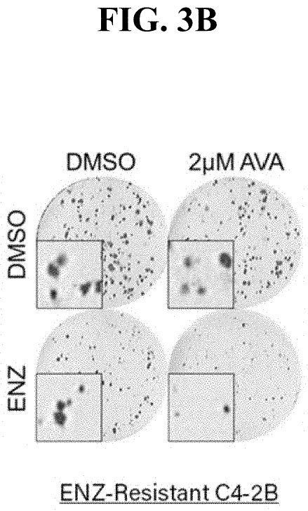

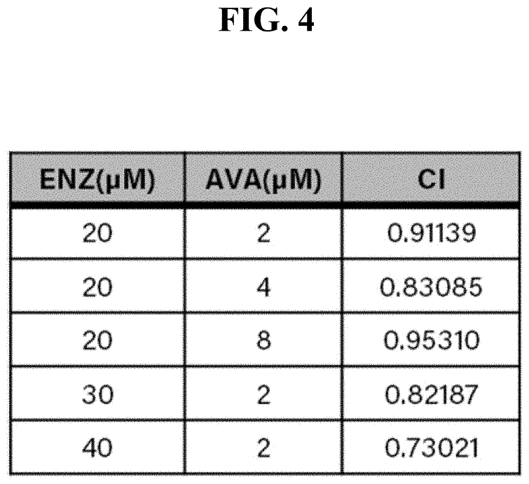

Pharmacological inhibition of SOAT1 has been shown to suppress tumor proliferation and metastasis of various cancer cells. However, its role in anti-androgen drug-resistant CRPC is unknown. To examine whether AVA, a pharmacological inhibitor of SOAT1, restores ENZ-sensitivity of ENZ-resistant CRPC cells, we performed a cell proliferation assay using 22RV1 cells treated with AVA, ENZ and a combination of AVA and ENZ. As shown in FIG. 1, 22RV1 cells were resistant to ENZ. Although the application of AVA slightly suppressed cell proliferation, 22RV1 cells were much more vulnerable to the combined treatment of ENZ and AVA. Moreover, we found that a combined treatment of AVA and ENZ sufficiently antagonized the clonogenic capacity of ENZ-resistant 22RV1 cells (FIGS. 2A and 2B) and C4-2B cells (FIGS. 3A and 3B) than any of the single treatments. To examine whether AVA and ENZ display a synergistic effect, we calculated the 50% combination index. According to our results, two drugs showed a synergistic effect (CI<1) in 22RV1 cells (FIG. 4). These data provide strong evidence that inhibition of SOAT1 has a synergistic effect with ENZ in ENZ-resistant CRPC cells.

### Example 3: Depletion of SOAT1 Mimics the Effect of SOAT1 Inhibitor and Offsets ENZ Resistance

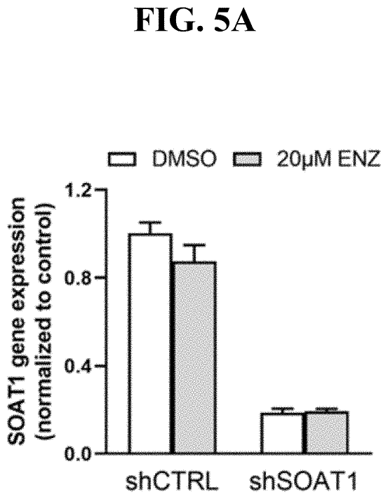

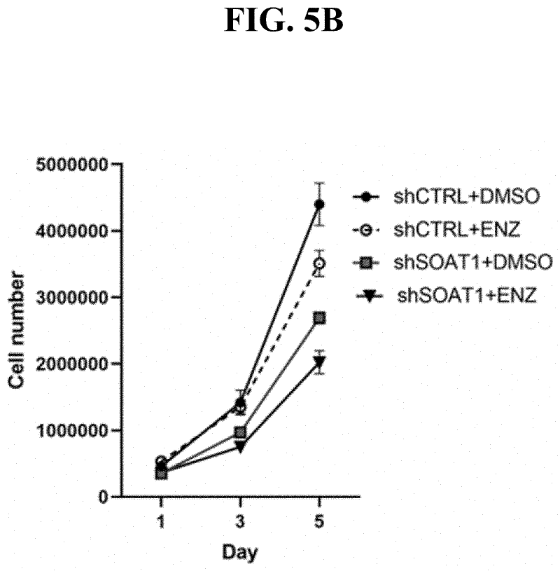

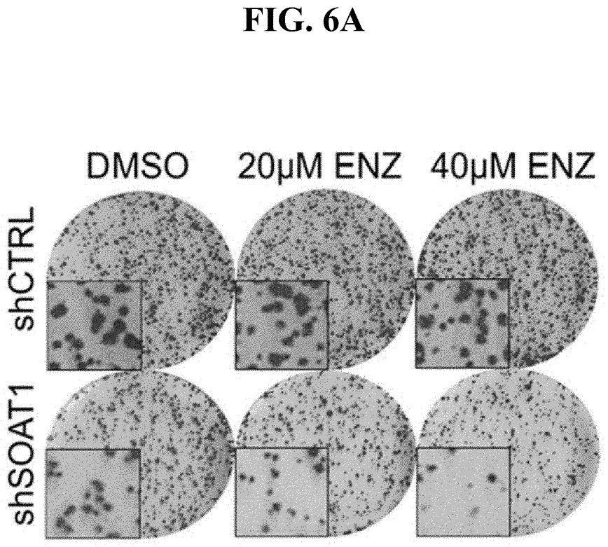

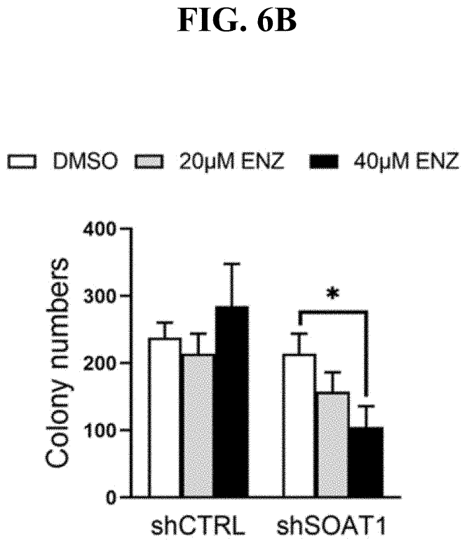

Next, we examined whether SOAT1 gene deficiency restores ENZ sensitivity of ENZ-resistant-CRPC cells. 22RV1 cells transfected with the shSOAT1 lentivirus resulted in an approximately 80% decrease in SOAT1 gene expression (FIG. 5A). Although ENZ treatment exhibited little effect on the proliferation of 22RV1 cells transfected with the shSOAT1 lentivirus, SOAT1 knockdown resulted in sensitization of 22RV1 cells to ENZ treatment (FIG. 5B). Consistently, SOAT1 knockdown antagonized the clonogenic capacity of ENZ-resistant 22RV1 cells (FIGS. 6A and 6B). These results provide that SOAT1 is likely to be a key regulator of ENZ resistance in 22RV1 cells.

### Example 4: Anti-Tumor Effect of the Combination Treatment of AVA and ENZ In Vivo

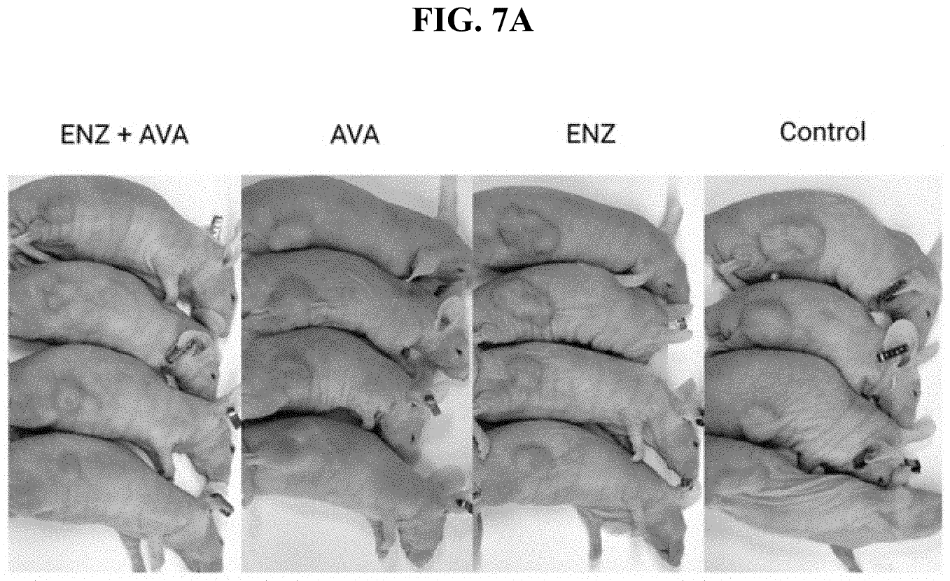

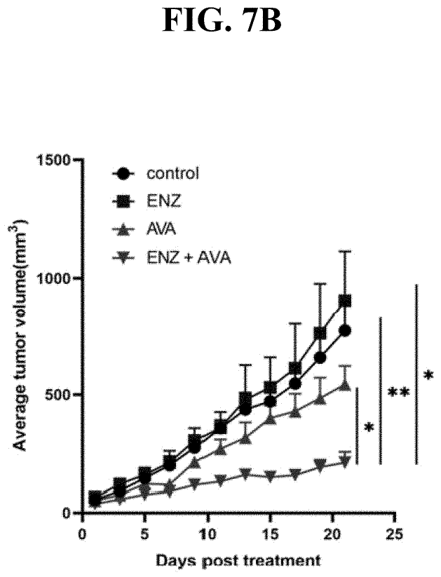

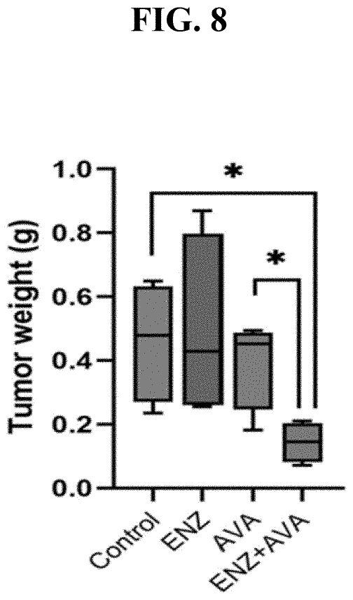

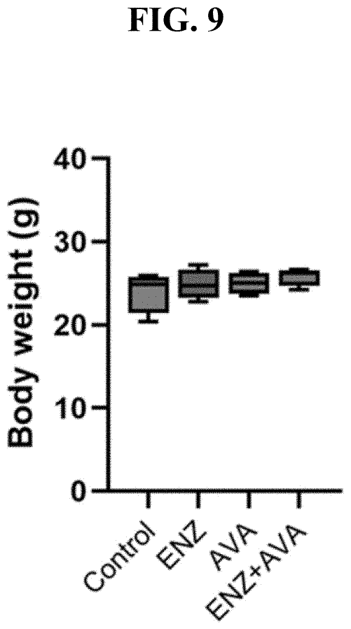

To further explore the translational potential of our finding, we performed an in vivo 22RV1 xenograft experiment. Consistent with our in vitro results, monotherapy with AVA or ENZ did not sufficiently delay the tumor growth as judged by the appearance of the tumor (FIG. 7A), and measurement of average tumor volume (FIG. 7B) and tumor weight (FIG. 8). In contrast, a combination treatment effectively decreased tumor growth. This is unlikely to result from the toxic effect of the combined therapy of AVA and ENZ, as the bodyweight of the mice exhibited no difference at the end of the study (FIG. 9). Collectively, these data provide additional evidence that inhibition of SOAT1 is sufficient to restore ENZ sensitivity, which offers a novel way to treat patients with ENZ-resistant CRPC.

### Example 5: High SOAT1 Expression is Correlated with AR Signaling Pathway, Myc and MTORC1 Signaling Pathway

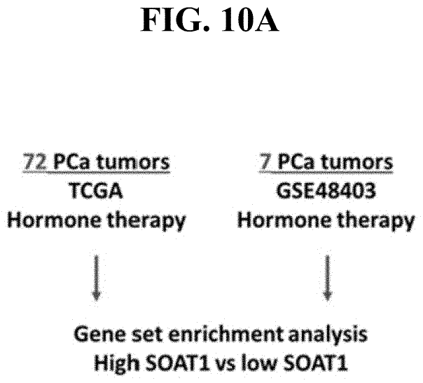

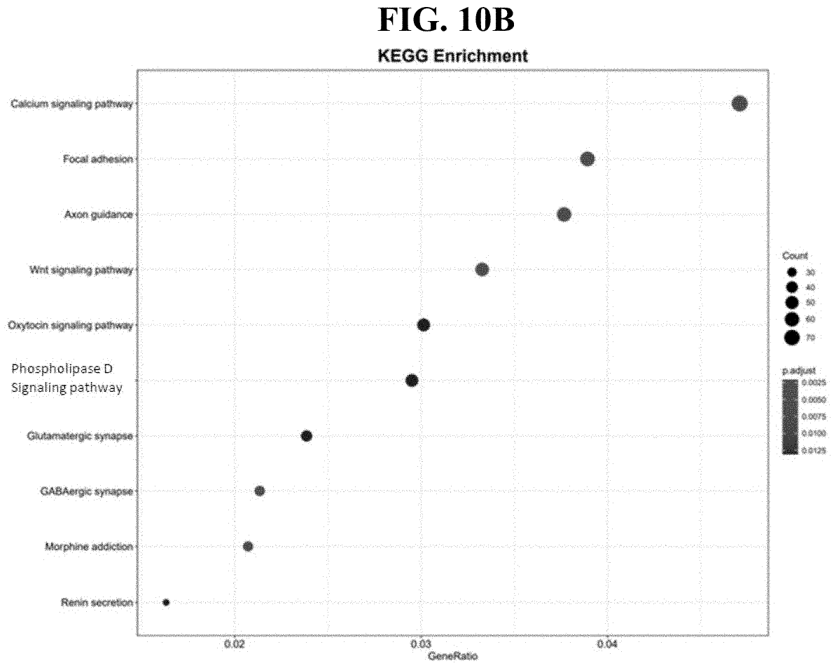

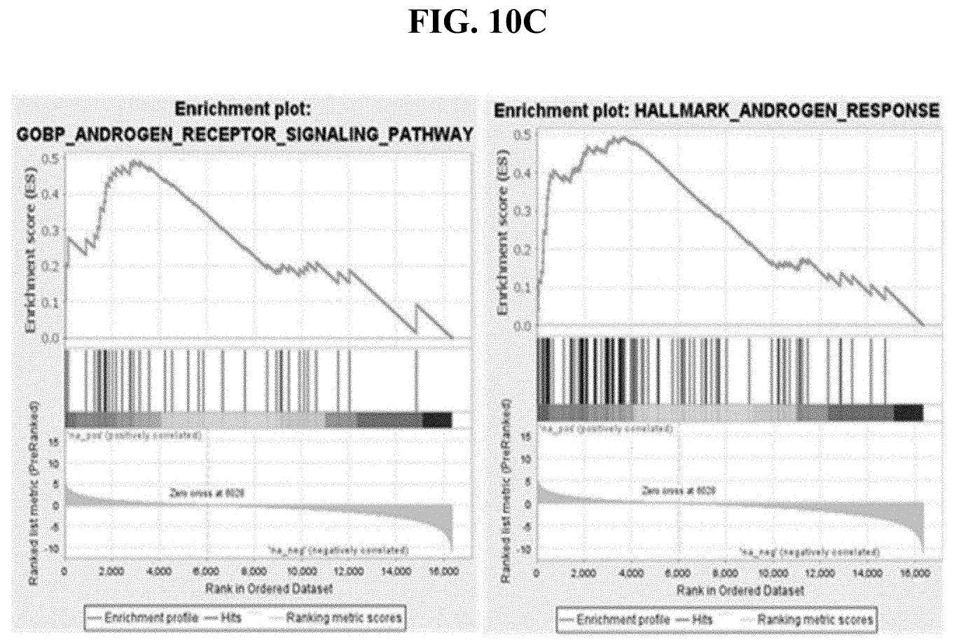

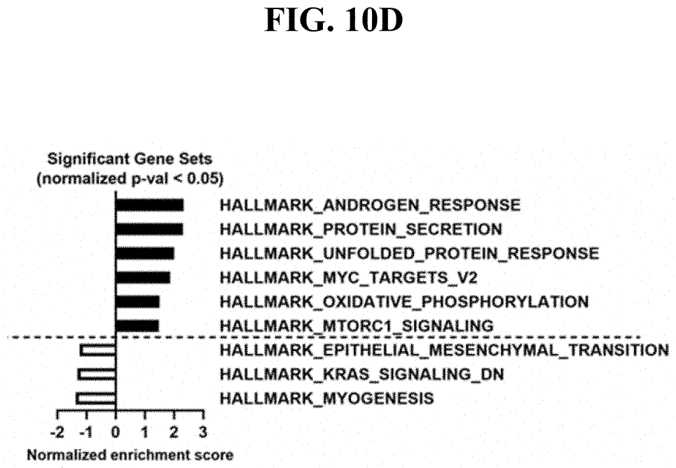

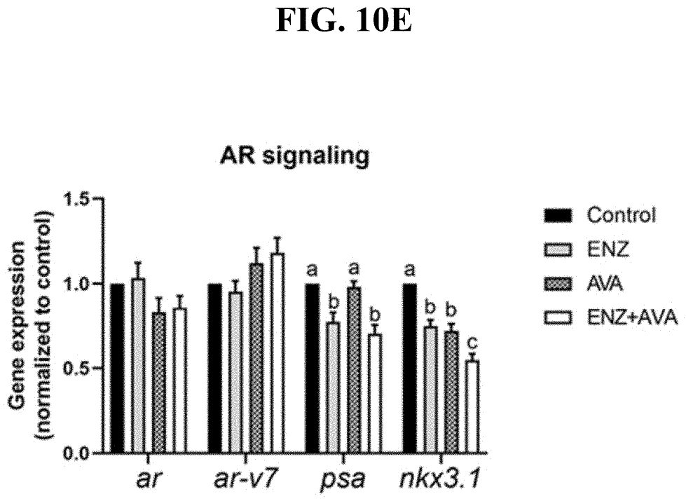

To investigate potential signaling pathways that SOAT1 is associated with, we analyzed RNA-Seq data of patients who had previously gone through antihormone therapy by comparing two groups divided by median cut-off (FIG. 10A). First, when differentially expressed genes were analyzed, Calcium signaling pathway, Focal adhesion, Axon guidance, Wnt signaling pathway, Oxytocin signaling pathway, Phospholipase D signaling pathway, Glutamatergic synapse, GABAergic synapse, Morphine addition and Renin secretion gene sets came up as top 10 enrichment (FIG. 10B). Interestingly, a previous study showed elevated phospholipase D activity and subsequent increase in phosphatidic acid promoted mTOR-dependent survival signal in hormone insensitive PCa cells [14]. We also ran GSEA with pre-ranked genes and found SOAT1 expression is highly correlated with AR signaling pathway (FIG. 10C). Other enriched gene sets include Protein secretion, Unfolded protein response, Myc targets, Oxidative phosphorylation and MTORC signaling pathway (FIG. 10D). To test whether the combination treatment could block the AR signaling, 22RV1 cells were treated with 20 μM ENZ in the presence or absence of 4 μM avasimibe for 24 h and mRNA levels associated with AR signaling were measured. AR and AR-V7 expression did not change but the downstream genes Psa and Nkx3.1 were downregulated by avasimibe (FIG. 10E).

Additional disclosure is found in Appendix-A, filed herewith, entirety of which is incorporated herein by reference into the present disclosure.

Those skilled in the art will recognize that numerous modifications can be made to the specific implementations described above. The implementations should not be limited to the particular limitations described. Other implementations may be possible.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### CITED REFERENCES

1. Sung, H.; Ferlay, J.; Siegel, R. L.; Laversanne, M.; Soerjomataram, I.; Jemal, A.; Bray, F. Global cancer statistics 2020: GLOBOCAN estimates of incidence and mortality worldwide for 36 cancers in 185 countries. CA Cancer J Clin 2021, doi:10.3322/caac.21660.
2. Crawford, E. D.; Petrylak, D.; Sartor, O. Navigating the evolving therapeutic landscape in advanced prostate cancer. Urol Oncol 2017, 35s, S1-s13, doi:10.1016/j.urolonc.2017.01.020.
3. Kirby, M.; Hirst, C.; Crawford, E. D. Characterising the castration-resistant prostate cancer population: a systematic review. Int J Clin Pract 2011, 65, 1180-1192, doi:10.1111/j.1742-1241.2011.02799.x.
4. Li, J.; Ren, S.; Piao, H. L.; Wang, F.; Yin, P.; Xu, C.; Lu, X.; Ye, G.; Shao, Y.; Yan, M., et al. Integration of lipidomics and transcriptomics unravels aberrant lipid metabolism and defines cholesteryl oleate as potential biomarker of prostate cancer. Sci Rep 2016, 6, 20984, doi:10.1038/srep20984.
5. Yue, S.; Li, J.; Lee, S. Y.; Lee, H. J.; Shao, T.; Song, B.; Cheng, L.; Masterson, T. A.; Liu, X.; Ratliff, T. L., et al. Cholesteryl ester accumulation induced by PTEN loss and PI3K/AKT activation underlies human prostate cancer aggressiveness. Cell Metab 2014, 19, 393-406, doi:10.1016/j.cmet.2014.01.019.
6. Liu, Y.; Wang, Y.; Hao, S.; Qin, Y.; Wu, Y. Knockdown of sterol O-acyltransferase 1 (SOAT1) suppresses SCD1-mediated lipogenesis and cancer procession in prostate cancer. Prostaglandins Other Lipid Mediat 2021, 153, 106537, doi:10.1016/j.prostaglandins.2021.106537.
7. Eckhardt, C.; Sbiera, I.; Krebs, M.; Sbiera, S.; Spahn, M.; Kneitz, B.; Joniau, S.; Fassnacht, M.; Kibler, H.; Weigand, I., et al. High expression of Sterol-O-Acyl transferase 1 (SOAT1), an enzyme involved in cholesterol metabolism, is associated with earlier biochemical recurrence in high risk prostate cancer. Prostate Cancer Prostatic Dis 2021, doi:10.1038/s41391-021-00431-3.
8. Nguyen, H. G.; Yang, J. C.; Kung, H. J.; Shi, X. B.; Tilki, D.; Lara, P. N.; DeVere White, R. W.; Gao, A. C.; Evans, C. P. Targeting autophagy overcomes Enzalutamide resistance in castration-resistant prostate cancer cells and improves therapeutic response in a xenograft model. Oncogene 2014, 33, 4521-4530, doi:10.1038/onc.2014.25.
9. Zhu, Y.; Kim, S. Q.; Zhang, Y.; Liu, Q.; Kim, K. H. Pharmacological inhibition of acyl-coenzyme A:cholesterol acyltransferase alleviates obesity and insulin resistance in diet-induced obese mice by regulating food intake. Metabolism 2021, 123, 154861, doi:10.1016/j.metabol.2021.154861.
10. Rajan, P.; Sudbery, I. M.; Villasevil, M. E.; Mui, E.; Fleming, J.; Davis, M.; Ahmad, I.; Edwards, J.; Sansom, O. J.; Sims, D., et al. Next-generation sequencing of advanced prostate cancer treated with androgen-deprivation therapy. Eur Urol 2014, 66, 32-39, doi:10.1016/j.eururo.2013.08.011.
11. Butler, L. M.; Centenera, M. M.; Swinnen, J. V. Androgen control of lipid metabolism in prostate cancer: novel insights and future applications. Endocr Relat Cancer 2016, 23, R219-227, doi:10.1530/ERC-15-0556.
12. Tousignant, K. D.; Rockstroh, A.; Taherian Fard, A.; Lehman, M. L.; Wang, C.; McPherson, S. J.; Philp, L. K.; Bartonicek, N.; Dinger, M. E.; Nelson, C. C., et al. Lipid Uptake Is an Androgen-Enhanced Lipid Supply Pathway Associated with Prostate Cancer Disease Progression and Bone Metastasis. Mol Cancer Res 2019, 17, 1166-1179, doi:10.1158/1541-7786. MCR-18-1147.
13. Tousignant, K. D.; Rockstroh, A.; Poad, B. L. J.; Talebi, A.; Young, R. S. E.; Taherian Fard, A.; Gupta, R.; Zang, T.; Wang, C.; Lehman, M. L., et al. Therapy-induced lipid uptake and remodeling underpin ferroptosis hypersensitivity in prostate cancer. Cancer Metab 2020, 8, 11, doi:10.1186/s40170-020-00217-6.
14. Utter, M.; Chakraborty, S.; Goren, L.; Feuser, L.; Zhu, Y. S.; Foster, D. A. Elevated phospholipase D activity in androgen-insensitive prostate cancer cells promotes both survival and metastatic phenotypes. Cancer Lett 2018, 423, 28-35, doi:10.1016/j.canlet.2018.03.006.

1. A method for treating or delaying castration-resistant prostate cancer (CRPC) comprising co-administering to a subject in need of relief from said cancer a therapeutically effective amount of a sterol-O-acyltransferase 1 (SOAT1) inhibitor together with a therapeutically effective amount of one or more inhibitors/antagonists of androgen receptor of androgen biosynthesis.

2. The method according to claim 1, wherein said androgen receptor inhibitor/antagonist comprises enzalutamide, bicalutamide, apalutamide, flutamide, nilutamide, darolutamide, and clascoterone, or a pharmaceutically acceptable salt thereof.

3. The method according to claim 1, wherein said androgen receptor inhibitor/antagonist is enzalutamide, or a pharmaceutically acceptable salt thereof.

4. The method according to claim 1, wherein said androgen biosynthesis inhibitor/antagonist is abiraterone, or a pharmaceutically acceptable salt thereof.

5. The method according to claim 1, wherein said CRPC is an androgen receptor inhibitor-resistant CRPC.

6. The method according to claim 5, wherein said CRPC androgen receptor inhibitor-resistant CRPC is an enzalutamide-resistant CRPC.

7. The method according to claim 1, wherein said CRPC is an androgen biosynthesis inhibitor-resistant CRPC.

8. The method according to claim 7, wherein said CRPC androgen biosynthesis antagonist-resistant CRPC is an abiraterone-resistant CRPC.

9. The method according to claim 1, wherein said SOAT1 inhibitor comprises avasimibe (CI-1011), CI-976, CP113,818, pactimibe, NTE-122, F-1394, PD140296, PD128042, PD132301-2, octimibate, DuP128, 58-035, HL-004, SMP-500, CL-277,082, SKF-99085, CS-505, eflucimibe (F12511), E5324, FR145237, CL277,082, YM-17E, FR129169, K-604, pyrocarbonate, beauveriolides I, and methanol extracts of Saururus chinensis root containing saucerneol B and manassantin B, or a pharmaceutically acceptable salt thereof.

10. The method according to claim 1, wherein said SOAT1 inhibitor is avasimibe, or a pharmaceutically acceptable salt thereof.

11. The method according to claim 1, wherein said androgen receptor inhibitor/antagonist and said SOAT1 inhibitor are combined first and administered consequently.

12. The method according to claim 1, wherein said androgen receptor inhibitor/antagonist and said SOAT1 inhibitor are formulated separately and administered consequently.

13. A pharmaceutical composition comprising a SOAT1 inhibitor and an inhibitor/antagonist of androgen receptor or androgen biosynthesis, together with one or more pharmaceutically acceptable diluents, excipients, or carriers.

14. The pharmaceutical composition according to claim 13, wherein said androgen receptor inhibitor/antagonist comprises enzalutamide, bicalutamide, apalutamide, flutamide, nilutamide, darolutamide, and clascoterone, or a pharmaceutically acceptable salt thereof.

15. The pharmaceutical composition according to claim 13, wherein said androgen receptor inhibitor/antagonist is enzalutamide, or a pharmaceutically acceptable salt thereof.

16. The pharmaceutical composition according to claim 13, wherein said androgen biosynthesis inhibitor/antagonist is abiraterone, or a pharmaceutically acceptable salt thereof.

17. The pharmaceutical composition according to claim 13, wherein said CRPC is an androgen receptor inhibitor-resistant CRPC.

18. The pharmaceutical composition according to claim 17, wherein said CRPC androgen receptor inhibitor-resistant CRPC is an enzalutamide-resistant CRPC.

19. The pharmaceutical composition according to claim 13, wherein said CRPC is an androgen biosynthesis inhibitor-resistant CRPC.

20. The pharmaceutical composition according to claim 19, wherein said CRPC androgen biosynthesis antagonist-resistant CRPC is an abiraterone-resistant CRPC.

21. The pharmaceutical composition according to claim 13, wherein said SOAT1 inhibitor comprises avasimibe (CI-1011), CI-976, CP113,818, pactimibe, NTE-122, F-1394, PD140296, PD128042, PD132301-2, octimibate, DuP128, 58-035, HL-004, SMP-500, CL-277,082, SKF-99085, CS-505, eflucimibe (F12511), E5324, FR145237, CL277,082, YM-17E, FR129169, K-604, pyrocarbonate, beauveriolides I, and methanol extracts of Saururus chinensis root containing saucerneol B and manassantin B, or a pharmaceutically acceptable salt thereof.

22. The pharmaceutical composition according to claim 13, wherein said SOAT1 inhibitor is avasimibe, or a pharmaceutically acceptable salt thereof.
